(19) United States
(12) Patent Application Publication
(10) Pub. No.: US 2009/0069633 A1
(43) Pub. Date: Mar. 12, 2009

(54) Capsule endoscope

(76) Inventors: Tatsuya Orihara, Shibuya-ku (JP); Hitoshi Fukuhori, Shibuya-Ku (JP)

Correspondence Address: Richard M. Rosati, Esq.;Kenyon & Kenyon LLP, One Broadway, New York, NY, 10004, US

(21) Appl. No.: 12/228,825

(22) Filed: Aug. 15, 2008

(30) Foreign Application Priority Data
Sep. 6, 2007 (JP) 2007-231362

(51) Int. Cl. A61B 1/06

(52) U.S. Cl. 600/163

(57) ABSTRACT

The invention relates to a capsule endoscope layout capable of achieving a small-format, wide-angle, wide light-distribution arrangement with limited variations. A capsule endoscope 1 comprises an objective lens 4, a transparent dome 2 to cover the object side of the objective lens, and light emitter devices located around the outer periphery of the objective lens. The endoscope 1 further comprises an integral-piece holder member 30 adapted to hold the objective lens 4 in place and hold the light emitter devices 5 at a position set back from an end of, and around, the objective lens 4 while the light emitter devices 5 are inclined outward at an angle with a center axis of said objective lens 4.

### BACKGROUND OF THE INVENTION

The present invention relates generally to a capsule endoscope, and more particularly to an-optimum structure for wide-field observation by capsule endoscopes.

Unlike conventional endoscopes, current capsule endoscopes have not a function of implementing in-vivo scans in any desired field direction; as compared with an endoscope having the same field range, a capsule endoscope would have a blind spot in a range incapable of changing the field of view, resulting in an increased probability of some oversight of lesions.

For this reason, the capsule endoscope having no function of implementing scans over the field range is designed to have a wide-angle, ahead-and-behind binocular imaging system (Patent Publication 1) so that any blind spot can be eliminated to stave off the oversight of lesions; the binocular imaging system is said to be a function of urgent need.

However, making the optical system a wide-angle arrangement leads to use of a number of lenses, resulting in total length increases and cost rises; there is a mounting demand toward achieving that wide-angle arrangement with as much reduced lens counts as possible (Patent Publication 2).

When a similar illumination system as used heretofore is employed while the range of field of an objective system is just widened, the range of field widened by the wide-angle arrangement is less brightly illuminated, possibly ending up with a drop of the rate of spotting lesions.

To take advantage of improvements in the performance due to the wide-angle arrangement for the imaging system, it is simultaneously necessary to distribute illumination light over a wider range (Patent Publications 3 and 4).
Patent Publication 1 Published Translation 2005-503182
Patent Publication 2 JP(A)2005-80713
Patent Publication 3 Internal Publication WO2004/096029
Patent Publication 4 JP(A)2004-275542

In view of such situations with the prior art as described, the present invention has for its object to provide a capsule endoscope layout capable of achieving a small-format, wide-angle, wide light-distribution arrangement with limited variations.

### SUMMARY OF THE INVENTION

According to the invention, the aforesaid object is accomplishable by the provision of a capsule endoscope which comprises an objective lens, a transparent dome to cover the object side of said objective lens, and light emitter devices located around the outer periphery of said objective lens, characterized by comprising an integral-piece holder member adapted to hold said objective lens in place and hold said light emitter device at a position set back from the end of, and around, said objective lens while said light emitter devices are inclined outward at an angle with the center axis of said objective lens.

Preferably in this case, the aforesaid holder member has a conical or pyramidal surface with an opening in a central portion thereof that is of the same shape as an external shape of said objective lens and an opening in a side thereof that is of the same shape as an external shape of each light emitting device, wherein said objective lens and each light emitter device are fitted and positioned in said openings.

That is, to incline the angle of the light emitter devices located, they are attached to a flexible substrate; without any holder structure, however, the location of the light emitter devices would get erratic. Unless the angle of inclination is kept with high precision, there would be variations in the distributed light, and flares as well. By use of the integral-piece holder member, the light emitter devices attached to the flexible substrate remain fixed with more reliable alignment of the objective lens with the light emitter devices so that there can be less variations in the distributed light, and less unwanted light.

If the objective lens is held by the integral-piece holder member, a portion with the light emitter devices attached to it is tapered to make the distributed light wide and the objective lens is located at the center of the endoscope, there is none of field shadings at the light emitter devices around the objective lens and at the portion with the light emitter device attached to it even when the objective lens has a wide angle of 180° or greater. Jutting out the objective lens in the transparent dome permits the objective lens to be located within the dome so that the total endoscope length can be curtailed at the same time.

The invention also provides a capsule endoscope which comprises an objective lens, a transparent dome to cover the object side of said objective lens, and light emitter devices located around the outer periphery of said objective lens, characterized in that:

there are two objective lenses provided ahead and behind, there is a transparent dome provided to cover the object sides of said objective lenses, there are light emitter devices provided around the outer peripheries of said objective lenses, and the light emitter devices provided ahead and behind are located at a position set back from the end of each object lens and inclined outward with respect to the center axis of each objective lens so that light distributed from the light emitter devices located ahead and behind intersects ahead and behind and around them.

To locate two wide-angle objective optical systems ahead and behind into a binocular arrangement for viewing images all around, the light emitter devices located ahead and behind should be such that the distributed light intersects ahead and behind and around them, thereby getting rid of portions that illumination light does not arrive at.

Preferably in the invention, the aforesaid objective lens has a field range of 140° or greater and satisfies the following condition (1):

0°<θ≦60° (1)

where θ is an angle that the center axis of each light emitter device in the radial direction makes with the center axis of the objective lens.

Preferably in the invention, the aforesaid objective lens has a three lenses arrangement comprising, in order from its object side, a meniscus lens having negative refracting power and convex on its object side, a lens having negative refracting power, a stop and a lens having positive refracting power.

The aforesaid light emitter device may be made up of a light emitting diode (LED), and an electroluminescent device (EL).

The former is bright and less costly. Attached to the flexible substrate, the latter is thin and may be attached even in narrow space, and is of fast response as well.

It is also desired to satisfy the following condition (2):

Ra>L (2)

where Ra is the radius of curvature of said object lens surface on the aforesaid transparent dome side, and L is the distance of the apex of the aforesaid objective lens surface on the aforesaid transparent dome side to the surface of the aforesaid objective lens nearest to the object side.

Thus, even when the objective lens makes its way into the transparent dome by way of a wide-angle objective system, the narrowing of the field does not occur so that the length of the whole capsule endoscope can be curtailed, making much contribution to easing off burdens on patients.

Further, the invention provides a capsule endoscope wherein the aforesaid light emitter devices are located such that when light is emitted out of each light emitter device onto a spherical object, light emitted out of the light emitter devices located ahead and behind and having an intensity of 10% or greater intersects assumed that the intensity of light emitted in the direction of the center axis of each light emitter device in the radial direction is 100%.

Yet further, the invention provides a capsule endoscope that satisfies the following condition (3):

(N/2)/tan(β−90°)≧(M/2)/tan(α+θ−90°) (3)

where N is a longitudinal distance between the centers of the light emitter devices located ahead and behind; M is a longitudinal distance between the centers of the ends of the objective lenses located ahead and behind; α is an angle with respect to the center axis at which there is a 10% intensity with respect to the intensity of light given out in the direction of the center axis of the light emitter device in the radial direction; β is a half the angle of field of the objective lenses; and θ is an angle that the center axes of the light emitter devices in the radial direction make with the center axes of the objective lenses.

According to the invention, the objective lenses are configured into a wide-angle arrangement, and the application of this arrangement to an illumination system layout for a conventional capsule endoscope would render the brightness of its periphery to be less sufficient, working against observations. However, the light emitter devices are inclined and located around the optical system so that it is possible to achieve a wide light-distribution illumination system compatible even with a wide-angle optical system, resulting in improvements in screening capability due to a wide-angle-of-field, wide light-distribution arrangement.

By locating two objective lenses each having an angle of field of 140° or greater in the ahead-and-behind direction to set up a binocular arrangement capable of viewing images nearly all around, it is possible to make substantial elimination of any blind spot. It is thus possible to get rid of blind spots even with a capsule endoscope having no function of changing the field direction freely and, hence, make improvements in screening capability.

Still other objects and advantages of the invention will in part be obvious and will in part be apparent from the specification.

The invention accordingly comprises the features of construction, combinations of elements, and arrangement of parts which will be exemplified in the construction hereinafter set forth, and the scope of the invention will be indicated in the claims.

### BRIEF DESCRIPTION OF THE DRAWINGS

FIG. 1 is illustrative in section of the construction of an end of the inventive capsule endoscope.

FIG. 2 is illustrative in section of a binocular type capsule endoscope wherein such end structures as shown in FIG. 1 are located ahead and behind.

FIG. 3 is illustrative in schematic of how the small intestine is observed and diagnosed inside by the capsule endoscope of FIG. 2.

FIG. 4(a) is illustrative in section of one exemplary construction of an end of the inventive capsule endoscope and FIG. 4(b) is a front view of the holder frame.

FIG. 5 is illustrative of one exemplary angle that the center axis of the light emitter device in the radial direction makes with the center axis of the objective lens, and how light is distributed then.

FIG. 6 is illustrative of how light is distributed from one exemplary light emitter device.

FIG. 7 is illustrative in schematic of the inventive capsule endoscope using the light emitter device of FIG. 6.

FIG. 8 is illustrative in section of an end structure of a prior art capsule endoscope.

FIG. 9 is illustrative in section of a prior art binocular type capsule endoscope.

### DESCRIPTION OF THE PREFERRED EMBODIMENTS

Examples of the inventive capsule endoscope are now explained with reference to the accompanying drawings.

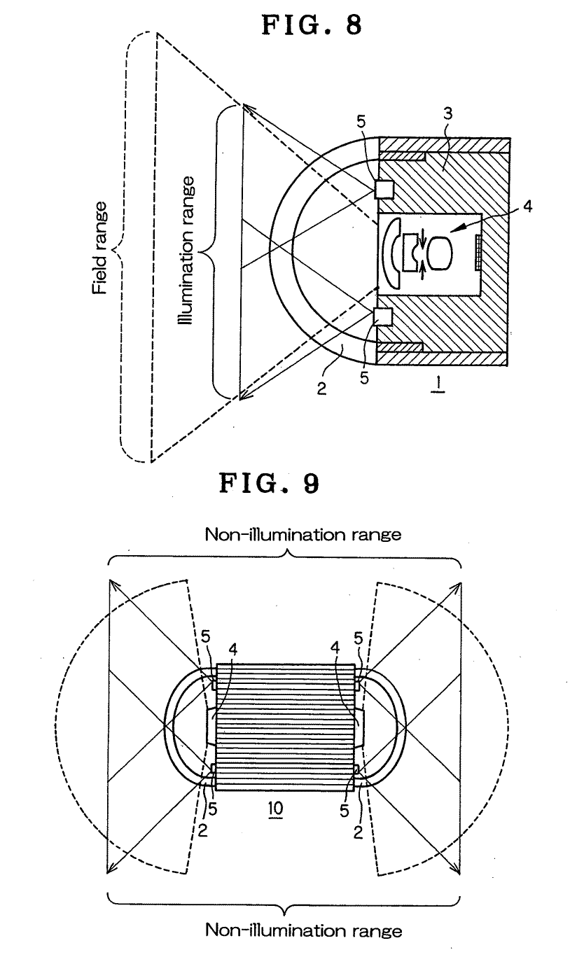

Referring to FIG. 8 that is illustrative of an end structure of a conventional capsule endoscope 1, a transparent, semispherical dome 2 is located over the end of the capsule endoscope 1; within the capsule endoscope 1 there is an objective lens 4 attached to the center of a frame member 3; and at a planar end of the frame member 3 around the objective lens 4 there are a plurality of light emitter devices 5 located symmetrically about the center axis, wherein each light emitter device comprises a light emitter diode (LED) or electroluminescent device (EL). In such arrangement, the field range (indicated by a broken line) of the objective lens 4 is included in the illumination range (indicated by a solid line) of the light emitter devices 5.

As end structures of such arrangement are located ahead and behind into a binocular type capsule endoscope 10, it causes a non-illumination range (area) to appear between the illumination ranges of both light emitter devices 5; even when the field range (broken line) of each objective lens 4 is widened, that non-illumination range renders brightness insufficient, resulting a drop of the rate of spotting lesions.

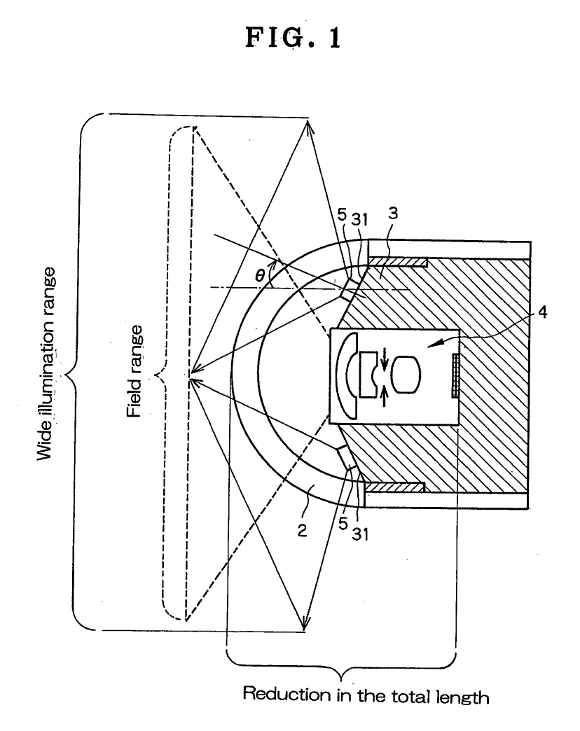

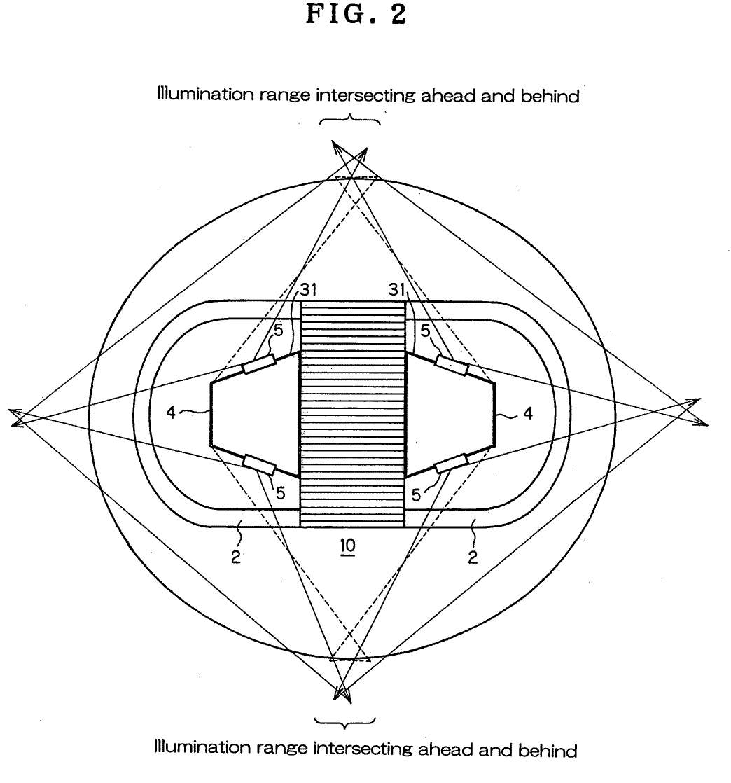

In the invention, therefore, there is a capsule endoscope 1 set up as shown in FIG. 1, wherein an end surface 31 of a frame member 3 around an objective lens 4 is configured as a conical or pyramidal shape such that an end of the centrally located objective lens 4 juts out in a dome 2 and positions of a plurality of light emitter devices 5 located around there are set back from an end of the objective lens 4, and the light emitter devices 5 are attached to that conical or pyramidal end surface 31 such that they direct outward obliquely with respect to the center axis of the objective lens 4. The range of illumination by the symmetrically located light emitter devices 5 grows wide and, with this, the field range of the objective lens 4 grows wide, resulting in a wider field range.

The field range of the objective lens 4 here is desirously 140° or greater. And in letting θ stand for an angle that the center axis of each light emitter device 5 in the radial direction makes with the center axis of the objective lens 4, it is desirous to satisfy the following condition.

0°<θ≦60° (1)

At the lower limit of 0° to condition (1), the same thing as in the prior art of FIG. 8 takes place: the endoscope runs short of illumination at off-axis sites with a drop of the rate of spotting lesions. As the upper limit of 60° is exceeded, on the contrary, the endoscope is likely to run short of illumination on the center axis.

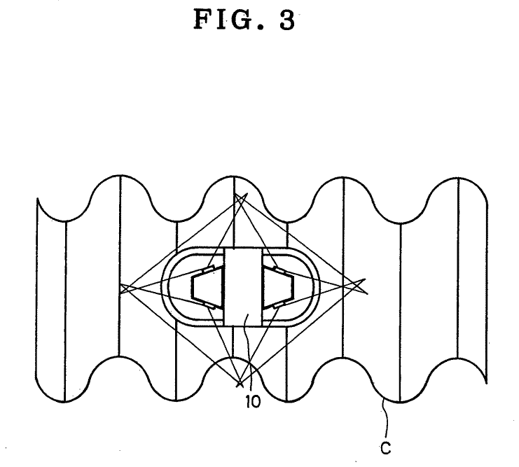

Referring now to a binocular type capsule endoscope 10 comprising such end structures as shown in FIG. 1 located ahead and behind, the illumination ranges by a plurality of light emitter devices 5 at the respective ends are set wider than 180° such that the ranges (illumination ranges) of light distributed from the light emitter devices 5 located ahead and behind intersect mutually (of course, the ranges of light distributed from a plurality of light emitter devices 5 located at the respective ends intersect mutually, too). This makes it possible to illuminate almost all around the capsule endoscope 10 so that, as shown schematically shown in FIG. 3, for instance, the small intestine C can be observed and diagnosed with no blind spot yet with a very low probability of losing sight of lesions.

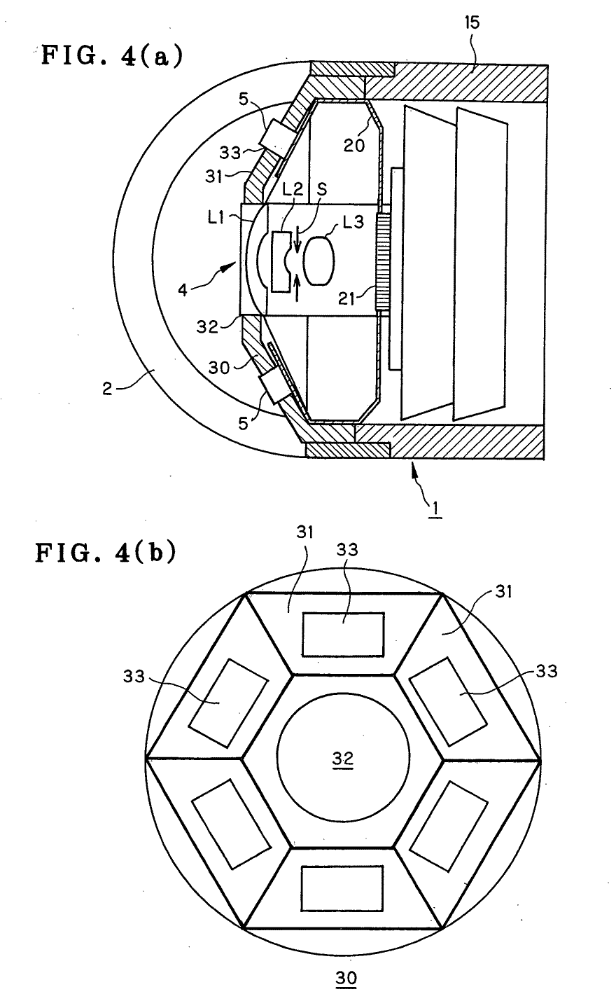

FIG. 4(a) is illustrative in section of one exemplary construction of one end of the capsule endoscope 1. At the end of a cylindrical housing 15 of the capsule endoscope, there is a holder frame 30 fixed, whose front view is presented in FIG. 4(b), and over that, a transparent, semispherical dome 2 is covered to form the end of the capsule endoscope 1. The holder frame 30 is formed of a sheet metal of hexagonal pyramid shape having in the center of an apex surface an opening 32 into which an objective lens 4 is fitted, and an opening 33 for fixing a light emitter device 5 is provided in one each side of the hexagonal pyramid. A lens barrel of the objective lens 4 is coaxially fixed in the opening 32 in the apex surface of the holder frame 30 of hexagonal pyramid shape, and a flexible substrate 20 having light emitter devices 5 at a given interval on its front surface is pressed against and fixed to the inside surface (back surface) of the holder frame 30, so that one each light emitter device 5 is inserted through and fixed in the opening 33 in one each side of the holder frame 30 from within the holder frame 30.

As set forth typically in Patent Publication 2, the objective lens 4 is of a three lenses type that is made up of, in order from its object side, a meniscus lens L1 having negative refracting power and convex on its object side, a lens L2 having negative refracting power, a stop S and a lens L3 having positive refracting power, so that the angle of field to be viewed can be set to 140° or greater, and preferably 180° or greater with no blind spot, leading to much less chances of losing sight of lesions. And an imaging device 21 such as CCD is located on the image plane of the objective lens 4 for connection to the flexible substrate 20.

Thus, by application to the end structure of the capsule endoscope 1 of the holder frame 30 capable of precisely determining the positions of location of the objective lens 4 and light emitter devices 5, it is possible to achieve a structure capable of holding the imaging system and the illumination system as an integral piece, thereby determining the location of the objective lens 4 and light emitter devices 5 with high precision. The light emitter devices 5 are mounted on the flexible substrate 20 that enables the directions of the devices to be freely determined, but without any holder structure, however, the location of the light emitter devices 5 would get erratic, possibly causing variations in light distribution, and flares. However, if, as described above, the integral-piece holder frame 30 is used which allows the light emitter devices 5 attached to the flexible substrate 20 to be fixed in place and the positions of the objective lens 4 and light emitter devices 5 to be determined with high precision, it is then possible to reduce the variations in light distribution and unwanted light, and improve assembly capabilities as well.

With the inventive capsule endoscope 1, the light emitter devices 5 are attached to the inclined sides of the holder frame 30 around the objective lens 4; the objective lens 4 is positioned jutting out in the transparent semispherical dome 2. It is thus possible just only to achieve a wide-field, wide light-distribution arrangement but also to reduce dead space in the dome 2 and curtail the length of the whole of the capsule endoscope 1 by the amount of jutting of the objective lens 4 into the dome 2. A reduction in the total length of the capsule endoscope 1 helps reduce burdens on patients and take hold of safety. To this end, it is desired to satisfy the following condition.

Ra>L (2)

Here Ra is the radius of curvature of the surface of the objective lens 4 on the transparent dome 2 side, and L is the distance from the apex of the surface of the objective lens 4 on the transparent dome 2 side to the surface located in, and nearest to the object side of, the objective lens (the object-side surface of the meniscus lens L1).

Beyond of the range of condition (2), the aforesaid effect on reductions of the total length is not obtainable.

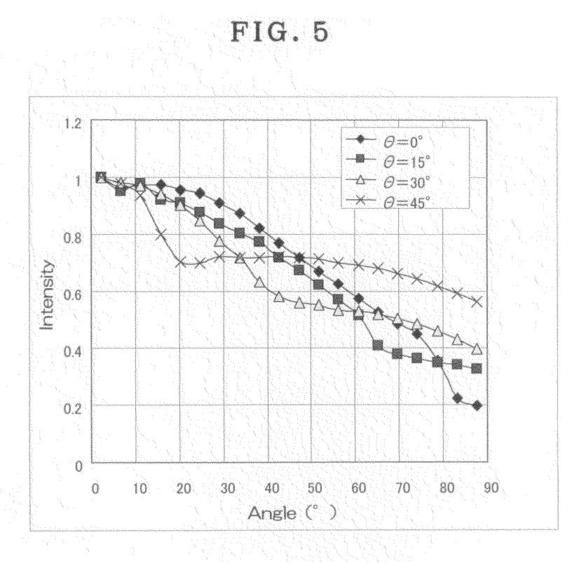

Referring then to FIG. 5, an example of the angle θ of the center axis of the light emitter device 5 in the radial direction with respect to the center axis of the objective lens 4 is shown together with light distributions in that case. A reference light distribution for each light emitter device 5 is obtained at θ=0, and light distributions at angles of 0 to 90° with the center axis of the objective lens 4 located at the angle θ of 15°, 30°, and 45° are drawn in FIG. 5. At the angle of location θ=45°, even the periphery of the objective lens 4 at 90° with respect to the center axis of the objective lens 4 is going to be brightly illuminated.

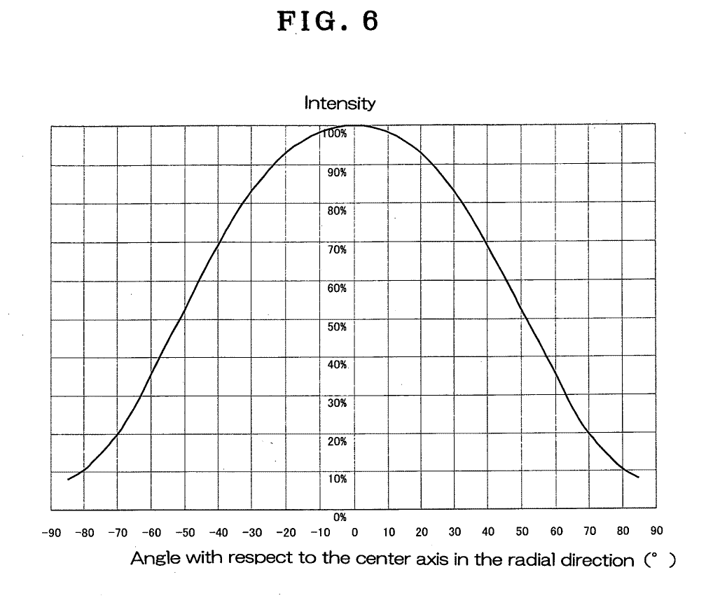

FIG. 6 is illustrative of light distributions for one example of a spherical form of light emitter device used here. The angle with respect to the center axis at which there is a 10% intensity with respect to the intensity of light given out in the direction of the center axis of the light emitter device in the radial direction is 80°.

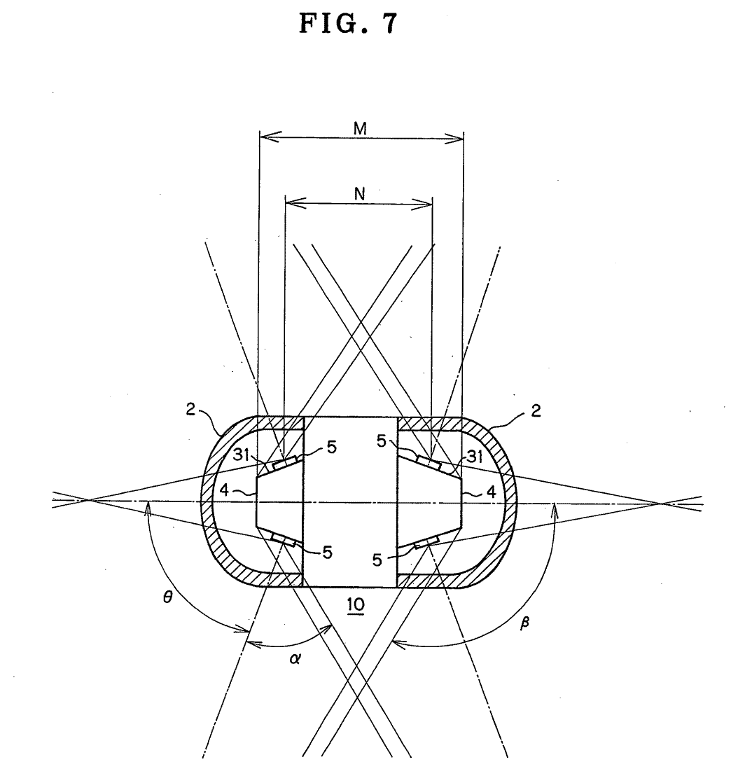

FIG. 7 is illustrative in schematic of the inventive capsule endoscope 10 with which that light emitter device 5 is used. A longitudinal distance N between the centers of light emitter devices 5 located ahead and behind is 10 mm; a longitudinal distance M between the centers of the ends of objective lenses 4 located ahead and behind is 11 mm; an angle a with respect to the center axis at which there is a 10% intensity with respect to the intensity of light given out in the direction of the center axis of the light emitter device 5 in the radial direction is 80°; an angle θ that the center axis of the light emitter device 5 in the radial direction makes with the center axis of the objective lens 4 is 35°; and a half β the angle of field of the objective lens 4 is 110°. That is,

(N/2)/tan(β−90°)=13.7

(M/2)/tan(α+θ−90°)=11.8

of which (N/2)/tan(β−90°)=13.7 is larger.

(N/2)/tan(β−90°)≧(M/2)/tan(α+θ−90°) (3)

By satisfying condition (3), the light given out of the light emitter device 5 falling within the field of view is supposed to have an intensity of 10% or greater; even when the objective lens has a wide-angle field, a bright image can be viewed as far as its periphery.

While the inventive capsule endoscope has been described with reference to its examples, it is appreciated that the invention is never limited to them: various modifications may be achievable.

What we claim is:

1. A capsule endoscope which comprises an objective lens, a transparent dome to cover an object side of said objective lens, and light emitter devices located around an outer periphery of said objective lens, characterized by comprising an integral-piece holder member adapted to hold said objective lens in place and hold said light emitter devices at a position set back from an end of, and around, said objective lens while said light emitter devices are inclined outward at an angle with a center axis of said objective lens.

2. The capsule endoscope according to claim 1, characterized in that said holder member has a conical or pyramidal surface with an opening in a central portion thereof that is of the same shape as an external shape of said objective lens and an opening in a side thereof that is of the same shape as an external shape of each light emitting device, wherein said objective lens and each light emitter device are fitted and positioned in said openings.

3. A capsule endoscope which comprises an objective lens, a transparent dome to cover the object side of said objective lens, and light emitter devices located around the outer periphery of said objective lens, characterized in that:
there are two objective lenses provided ahead and behind, there is a transparent dome provided to the object sides of said objective lenses, there are light emitter devices provided around the outer peripheries of said objective lenses, and the light emitter devices located ahead and behind are located at a position set back from the end of each object lens and inclined outward with respect to the center axis of each objective lens so that light distributed from the light emitter devices located ahead and behind intersects ahead and behind and around them.

4. The capsule endoscope according to claim 1, wherein said objective lens has a field range of 140° or greater and satisfies the following condition (1):
0°<θ≦60° (1)
where θ is an angle that a center axis of each light emitter device in a radial direction makes with a center axis of the objective lens.

5. The capsule endoscope according to claim 4, wherein each objective lens comprises a three lenses arrangement comprising, in order from an object side thereof, a meniscus lens having negative refracting power and convex on an object side thereof, a lens having negative refracting power, a stop and a lens having positive refracting power.

6. The capsule endoscope according to claim 3, wherein each objective lens has a field range of 140° or greater and satisfies the following condition (1):
0°<θ≦60° (1)
where θ is an angle that a center axis of each light emitter device in a radial direction makes with a center axis of each objective lens.

7. The capsule endoscope according to claim 6, wherein each objective lens comprises a three lenses arrangement comprising, in order from an object side thereof, a meniscus lens having negative refracting power and convex on an object side thereof, a lens having negative refracting power, a stop and a lens having positive refracting power.

8. The capsule endoscope according to claim 1, characterized in that each light emitter device comprises a light-emitting diode (LED).

9. The capsule endoscope according to claim 3, characterized in that each light emitter device comprises a light-emitting diode (LED).

10. The capsule endoscope according to claim 1, characterized in that each light emitter device comprises an electroluminescent device (EL).

11. The capsule endoscope according to claim 3, characterized in that each light emitter device comprises an electroluminescent device (EL).

12. The capsule endoscope according to claim 1, characterized by satisfying the following condition (2):
Ra>L (2)
where Ra is a radius of curvature of an object lens surface covered by said transparent dome, and L is a distance of an apex of said objective lens surface covered by said transparent dome to a surface of said objective lens nearest to an object side thereof.

13. The capsule endoscope according to claim 3, characterized by satisfying the following condition (2):
Ra>L (2)
where Ra is a radius of curvature of an object lens surface covered by said transparent dome, and L is a distance of an apex of said objective lens surface covered by said transparent dome to a surface of said objective lens nearest to an object side thereof.

14. The capsule endoscope according to claim 3, characterized in that said light emitter devices are located such that when light is emitted out of each light emitter device onto a spherical object, light emitted out of the light emitter devices located ahead and behind and having an intensity of 10% or greater intersects assumed that the intensity of light emitted in the direction of the center axis of each light emitter device in the radial direction is 100%.

15. The capsule endoscope according to claim 3, characterized by satisfying the following condition (3):
(N/2)/tan(β−90°)≧(M/2)/tan(α+θ−90°) (3)
where N is a longitudinal distance between the centers of the light emitter devices located ahead and behind; M is a longitudinal distance between the centers of the ends of the objective lenses located ahead and behind; α is an angle with respect to the center axis at which there is a 10% intensity with respect to the intensity of light given out in the direction of the center axis of the light emitter device in the radial direction; β is a half the angle of field of the objective lenses; and θ is an angle that the center axes of the light emitter devices in the radial direction make with the center axes of the objective lenses.
